(19) United States
(12) Patent Application Publication
(10) Pub. No.: US 2011/0294117 A1
(43) Pub. Date: Dec. 1, 2011

(54) NUCLEIC ACID SEQUENCING DEVICE AND METHOD OF DETERMINING NUCLEOTIDE SEQUENCE OF TARGET NUCLEIC ACID USING THE SAME

(75) Inventors: Suhyeon KIM, Seoul (KR); Joo-won RHEE, Yongsin-si (KR)

(73) Assignee: SAMSUNG ELECTRONICS CO., LTD, Suwon-si (KR)

(21) Appl. No.: 12/953,935

(22) Filed: Nov. 24, 2010

(30) Foreign Application Priority Data
Jun. 1, 2010 (KR) 10-2010-0051972

(51) Int. Cl. C12Q 1/68; G01N 21/65; G01N 21/64; G01N 27/00; G01N 27/02

(52) U.S. Cl. 435/6.11; 436/94; 422/82.01; 422/82.02; 422/82.06; 422/82.07; 435/6.19

(57) ABSTRACT

A nucleic acid sequencing device includes at least one nanochannel, a first electrode and a second electrode disposed at opposite ends of the nanochannel for applying a voltage in the lengthwise direction of the nanochannel, and a first detector that detects a location signal of a target nucleic acid passing through the nanochannel and a second detector that detects a signal from a detectable label bound to the target nucleic acid.

### CROSS-REFERENCE TO RELATED APPLICATIONS

This application claims priority to Korean Patent Application No. 10-2010-0051972, filed on Jun. 1, 2010, and all the benefits accruing therefrom under 35 U.S.C. §119, the content of which in its entirety is herein incorporated by reference.

### BACKGROUND

1. Field

The present disclosure relates to a nucleic acid sequencing device and a method of determining a nucleotide sequence of a target nucleic acid using the same.

2. Description of the Related Art

A gene is made up of a linear alignment of four types of nucleotides which are distinguished from each other by a base. The different bases are adenine, cytosine, guanine, and thymine. Two of the most popular gene sequencing techniques are a chain termination method and a chemical degradation method. However, theses methods are cost- and effort-consuming processes because only a limited size of the nucleotide sequence of DNA can be determined at once. As a result these methods and not suitable for sequencing high-volume target sequences, on projects such as, for example, the human genome project. By using a next generation sequencing (“NGS”) technique introduced in 2005 which does not use the chain termination method, the volume of gene sequencing is rapidly increased, and costs for sequencing genes is significantly reduced.

The NGS techniques are classified into second-generation sequencing and third-generation sequencing. Second-generation sequencing uses DNA clones in a cycling reaction. This is expensive and therefore less used as compared with third-generation sequencing. Third-generation sequencing uses a single DNA as a result of which the sequencing process may be conducted in various ways. Sequencing using nanopores is the most efficient method among the third-generation sequencing techniques. However, a single DNA molecule passes through the nanopores too quickly to provide sufficient detection time for determining the nucleotide sequence of DNA.

There is therefore a need to develop a method and a device for efficiently determining the nucleotide sequence of a target nucleic acid.

### SUMMARY

Provided are a nucleic acid sequencing device including at least one nanochannel and a method of determining a nucleotide sequence of a target nucleic acid using the nucleic acid sequencing device.

Additional aspects will be set forth in part in the description which follows and, in part, will be apparent from the description, or may be learned by practice of the presented embodiments.

According to an aspect of the present invention, there is provided a nucleic acid sequencing device including at least one nanochannel.

According to another aspect of the present invention, there is provided a method of determining a nucleotide sequence of a target nucleic acid using the nucleic acid sequencing device.

A nucleic acid sequencing device comprises at least one nanochannel, a first electrode and a second electrode are disposed at both opposite ends of the nanochannel for applying a voltage in the lengthwise direction of the nanochannel, and a first detector that detects a location signal of a target nucleic acid passing through the nanochannel and a second detector that detects a signal from a detectable label linked to the target nucleic acid.

Disclosed herein too is a method of determining a nucleotide sequence of a target nucleic acid, the method comprising linking a nanoparticle comprising a detectable label to a 5′ or 3′ end of a target nucleic acid having a nucleotide sequence to be detected; making the target nucleic acid contact a probe comprising a detectable label; injecting the target nucleic acid into a nanochannel; applying a voltage between the first electrode and the second electrode of the nucleic acid sequencing device; and detecting a signal generated from the nanoparticle linked to the target nucleic acid and a signal generated from the detectable label of the probe linked to the target nucleic acid.

### BRIEF DESCRIPTION OF THE DRAWINGS

The above and other aspects, advantages and features of this disclosure will become more apparent by describing in further detail exemplary embodiments thereof with reference to the accompanying drawings, in which:

FIG. 1 is a schematic diagram of a nucleic acid sequencing device for determining a nucleotide sequence of a target nucleic acid according to an embodiment of the present invention;

FIG. 2 is a schematic diagram for describing a method of determining a nucleotide sequence of a target nucleic acid using a nucleic acid sequencing device according to an embodiment of the present invention;

FIGS. 3A and 3B are schematic diagrams for describing a method of measuring a location of a nanoparticle linked to an end of a target nucleic acid, by using an image sensor as a first detector disposed in the nucleic acid sequencing device according to an embodiment of the present invention;

FIG. 4 is a schematic diagram for describing a method of predicting a location of a nanoparticle by using a nanopattern disposed the nucleic acid sequencing device according to an embodiment of the present invention;

FIG. 5 is a schematic diagram for describing a method of converting a signal in a converter in order to measure a location of a nanoparticle linked to a target nucleic acid in the nucleic acid sequencing device according to an embodiment of the present invention; and

FIGS. 6A and 6B are schematic diagrams for describing a method of improving precision of the measurement of a location of a nanoparticle linked to a target nucleic acid in the nucleic acid sequencing device according to an embodiment of the present invention.

### DETAILED DESCRIPTION

The invention now will be described more fully hereinafter with reference to the accompanying drawings, in which various embodiments are shown. This invention may, however, be embodied in many different forms, and should not be construed as limited to the embodiments set forth herein. Rather, these embodiments are provided so that this disclosure will be thorough and complete, and will fully convey the scope of the invention to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein. Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims. In one embodiment, a nucleic acid sequencing device includes at least one nanochannel, a first electrode and a second electrode that are disposed at both ends of the nanochannel for applying a voltage in the lengthwise direction of the nanochannel, and a first detector that detects a location signal of a target nucleic acid passing through the nanochannel and a second detector that detects a signal from a detectable label bound to the target nucleic acid. The first detector and the second detector may be disposed in the nanochannel.

The nucleic acid sequencing device is a microfluidic device including at least one nanochannel. The “microfluidic device” used herein refers to a device including at least one inlet and outlet which are connected to each other via a nanochannel. In general, the microfluidic device includes a nanochannel or chamber for constant chemical reaction or analysis. The channel may have various shapes of cross-section, for example, circular, rectangular, or trapezoid cross-section, but is not limited thereto.

According to an embodiment, the width or depth of the nanochannel may be in the range of about 10 to about 1,000 nanometers (“nm”), for example, about 10 to about 100 nm. The nanochannel has openings at both ends, and the target nucleic acid may pass through the nanochannel from one opening to the other opening.

The “target nucleic acid” used herein refers to polynucleotide having a nucleotide sequence to be analyzed. The nucleic acid is widely used to indicate DNA (“gDNA” and “cDNA”) and/or RNA, peptide nucleic acid (“PNA”), and locked nucleic acid (“LNA”). A nucleotide, which is a basic unit of nucleic acid, includes not only natural nucleotides but also nucleotide analogues in which a sugar or base is modified. In addition, a mediator that mediates the transfer of the target nucleic acid may be used in order to transfer the target nucleic acid through the nanochannel. A buffer solution that is known in the art may be used as the mediator.

In another embodiment, a first electrode and a second electrode are disposed in the nanochannel for applying a voltage in the lengthwise direction of the nanochannel. The first and second electrodes may be disposed at both ends of the microfluidic device. In addition, a voltage applied to the first electrode may have an opposite polarity to a voltage applied to the second electrode. For example, when a target material such as nucleic acid having negative polarity flows into the nanochannel, the application of a voltage may be controlled such that the first electrode has negative polarity and the second electrode has positive polarity. In addition, the device may further include a voltage controller that controls the intensity or the magnitude of polarity. The velocity of the target material flowing in the nanochannel may be controlled by the voltage controller.

According to an embodiment, a first detector that detects a location signal of the target nucleic acid passing through the nanochannel may be disposed in the nanochannel. A second detector that detects a signal from a detectable label bound to the target nucleic acid may also be disposed in the nanochannel.

The first and/or second detector may be an optical detector or an electrical detector. The electrical detector may detect at least one selected from the group consisting of a current, voltage, resistance, and impedance, and the optical detector may detect at least one selected from the group consisting of an absorbance, transmission, scattering, fluorescence, fluorescence resonance energy transfer (“FRET”), surface plasmon resonance, surface enhanced Raman scattering, and diffraction.

According to an embodiment, the device may further include a sample inlet and a sample outlet, which are in fluid communication with the openings of both ends of the nanochannel. The sample inlet may include a microfluidic region including at least one microfluidic channel and a nanofluidic region including at least one nanofluidic channel so as to allow fluid flow therebetween. Since the sample inlet may include at least one microfluidic channel and nanofluidic channel which are disposed in a direction such that the diameter of the nanochannel decreases, a sample injected may be spread while passing through the channels disposed in the sample inlet by its structural limit. Finally, the concentration of the target material is adjusted so that a single molecule of the target material may flow into the nanochannel of the nucleic acid sequencing device at once.

According to an embodiment, the device may further include a converter that converts the signal detected by the first detector into location information of one end of the target nucleic acid and the signal detected by the second detector into relative location information of the probes of the target nucleic acid. The device may further includes a calculator that determines a nucleotide sequence of the target nucleic acid based on the information obtained from the converter.

The device may further include an output unit that outputs the nucleotide sequence of the target nucleic acid determined by the calculator to a user.

According to another embodiment, there is provided a method of determining a nucleotide sequence of a target nucleic acid, the method including linking a nanoparticle including a detectable label to a 5′ or 3′ end of a target nucleic acid having a nucleotide sequence to be detected; making the target nucleic acid contact a probe including a detectable label; injecting the target nucleic acid into the nanochannel; applying a voltage between the first electrode and the second electrode of the nucleic acid sequencing device; and detecting a signal generated from the nanoparticle linked to the target nucleic acid and a signal generated from the detectable label of the probe bound to the target nucleic acid.

The method of determining a nucleotide sequence of a target nucleic acid will now be described in more detail.

The method includes linking a nanoparticle including a detectable label to a 5′ or 3′ end of a target nucleic acid having a nucleotide sequence to be detected.

The “nanoparticle” used herein refers to a particle having a diameter of about 1 to 200 nm. Components of the nanoparticle may include a metal such as gold, silver, copper, aluminum, nickel, palladium, and platinum, a semiconductor material such as cadmium selenide (CdSe), cadmium sulfide (CdS), indium arsenide (InAs), and indium phosphide (InP), and an inert material such as polystyrene, latex, acrylate, polypeptide, or the like, or a combination thereof.

The nanoparticle and the target nucleic acid may be linked to each other by a 1:1 covalent bond. For this, the nanoparticle may be chemically linked to the target nucleic acid, and the target nucleic acid linked to the nanoparticle may be isolated from an unlinked target nucleic acid or an unlinked nanoparticle. For example, the target nucleic acid linked to the nanoparticle may be isolated using magnetic characteristics of the nanoparticle, difference of electrophoresis rate, or physical characteristics such as size by adding excess target nucleic acid. A single functional group that is linkable to the nanoparticle may also be used. When a polypeptide is used, a functional group at a C- or N-terminal may be used. If the nanoparticle is a bead-shaped nanoparticle having more than one functional group, a single functional group among them may be used for the linkage. For example, a method of linking an oligo nucleic acid having a modified nucleotide at its end to a nanoparticle is disclosed by Nam et al., Nature Material vol 9, 60 p (2010). The same effects as described above may be obtained by introducing a functional group into the oligo nucleic acid. In addition, in a double-stranded DNA, reaction by the functional group may occur at both ends, and thus DNA linked to the nanoparticle is isolated from DNAs and nanoparticles that are not linked using additional processes.

The nanoparticle may be linked to a 5′ end or 3′ end of the target nucleic acid in order to enhance the stretch of DNA by accelerating or decelerating the transfer of the target nucleic acid in the microfluidic device. In addition, the nanoparticle may include a detectable label so that the location of the target nucleic acid migrating within the nanochannel may be detected.

The “detectable label” used herein refers to an atom, molecule, or particle used to specifically detect a molecule including the label among the same type of molecules without the label. For example, the detectable label may include colored beads, an antigen determinant, an enzyme, a hybridizable nucleic acid, a chromophore, a fluorescent material, an electrically detectable material, a material providing modified fluorescence-polarization or modified light-diffusion, and/or a quantum dot. In addition, the detectable label may include a labeled binding protein, a heavy metal atom, a spectroscopic marker such as a dye, or a magnetic label.

The dye may be quinoline dyes and their derivatives, triarylmethane dyes and their derivatives, phthalenes and their derivatives, azo dyes and their derivatives, or cyanine dyes and their derivatives, anthranones and their derivatives; anthraquinones and their derivatives; croconines and their derivatives; monoazos, disazos, trisazos and their derivatives; benzimidazolones and their derivatives; diketo pyrrole pyrroles and their derivatives; dioxazines and their derivatives; diarylides and their derivatives; indanthrones and their derivatives; isoindolines and their derivatives; isoindolinones and their derivatives; naphtols and their derivatives; perinones and their derivatives; perylenes and their derivatives such as perylenic acid anhydride or perylenic acid imide; ansanthrones and their derivative; dibenzpyrenequinones and their derivatives; pyranthrones and their derivatives; bioranthorones and their derivatives; isobioranthorone and their derivatives; diphenylmethane, and triphenylmethane, type pigments; cyanine and azomethine type pigments; indigoid type pigments; bisbenzoimidazole type pigments; azulenium salts; pyrylium salts; thiapyrylium salts; benzopyrylium salts; phthalocyanines and their derivatives, pryanthrones and their derivatives; quinacidones and their derivatives; quinophthalones and their derivatives; squaraines and their derivatives; squarilylums and their derivatives; leuco dyes and their derivatives, deuterated leuco dyes and their derivatives; leuco-azine dyes; acridines; di- and tri-arylmethane, dyes; quinoneamines; o-nitro-substituted arylidene dyes, aryl nitrone dyes, or the like, or a combination comprising at least one of the foregoing.

The fluorescent material may be a fluorescein and its derivatives, a phycoerythrin and its derivatives, a rhodamine and its derivatives, a lissamine and its derivatives, coumarin and its derivatives, phycocyanin and its derivatives, allophycocyanin and its derivatives, o-phthaldehyde and its derivatives, fluorescamine and its derivatives, Cy3 or Cy5 (Pharmacia), or the like, or a combination comprising at least one of the foregoing fluorescent materials. An example of a suitable fluorescent material is DAPI (4′,6-diamidino-2-phenylindole) that binds strongly to DNA.

Meanwhile, the diameter of the nanoparticle may reduce the velocity of the target nucleic acid within the nanochannel. The nanoparticle may have a diameter suitable for passing through the nanochannel, for example, in the range of about 1 to about 200 nm, specifically about 2 to about 100 nm and more specifically about 4 to about 10 nm. In addition, the nanoparticle may have the same polarity as a voltage applied to the first electrode so that the target nucleic acid strands linked to the nanoparticle are stretched in the nanochannel. In this regard, the mobility of the nanoparticle should be less than that of the target nucleic acid in a applied voltage so that the target nucleic acid strands linked to the nanoparticle may migrate ahead in the nanochannel). In addition, the target nucleic acid is single-stranded or double stranded.

The method includes making the target nucleic acid contact a probe including a detectable label.

The contact between the target nucleic acid and the probe may be conducted in vitro under stringent conditions that are known in the art and in a suitable buffer solution.

The “probe” used herein refers to a nucleic acid or protein that is linkable to a target nucleic acid having a complementary sequence by at least one chemical bond, generally complementary base paring, such as, for example, hydrogen bond between bases. The probe may be a nucleic acid or protein that is complementary to a part of the nucleotide sequence of the target nucleic acid. If the probe is a nucleic acid, it may generally have about 3 to about 100 nucleotides, specifically about 4 to about 50 nucleotides. The nucleic acid may be DNA, RNA, PNA, or LNA.

If the probe is a protein, the sequence of amino acid that specifically recognizes the sequence of the target nucleic acid in the target sequence-binding protein may include a nucleic acid-binding motif, and the protein may include at least one nucleic acid-binding motif. According to an embodiment, the sequence of the amino acid that is specifically bound to the sequence of the target nucleic acid may include at least one nucleic acid-binding motif selected from the group consisting of zinc finger motif, helix-turn-helix motif, helix-loop-helix motif, leucine zipper motif, nucleic acid-binding motif of restriction endonuclease, and combinations thereof. For example, zinc finger motifs may specifically recognize different nucleotide sequences, a nucleotide sequence specifically recognized by the amino acid sequence of the zinc finger motif is disclosed in http://www.scripps.edu/mb/barbas/zfdesign/zfdesignhome.php. The probe may include a detectable label, the detectable label is described above.

The method includes injecting the target nucleic acid linked to the nanoparticle into a nanochannel of the nucleic acid sequencing device.

For example, the target nucleic acid may be injected via a sample inlet disposed in the nucleic acid sequencing device automatically using a sample injecting device (e.g., pump) or manually by an experimenter. If the target nucleic acid is injected through the sample inlet, a single strand of a nucleic acid molecule may be injected into the nanochannel.

The method includes applying a voltage between the first electrode and the second electrode of the nucleic acid sequencing device.

In this regard, the voltage applied to the first electrode may have an opposite polarity to the voltage applied to the second electrode. That is, since the target nucleic acid has negative polarity, the voltage may be applied to the first and second electrodes such that the first electrode has negative polarity and the second electrode has positive polarity in a microfluidic device according to an embodiment. Since the voltage is applied as described above, the target nucleic acid flows in the nanochannel and migrates in a direction toward the second electrode because of its opposing polarity.

The method includes detecting a signal generated from the nanoparticle linked to the target nucleic acid and a signal generated from the detectable label of the probe bound to the target nucleic acid.

The signal generated from the nanoparticle that includes a detectable label that is bound to the target nucleic acid and may be detected by the first detector of the nucleic acid sequencing device. The signal may vary according to the types of the detectable label, and examples thereof are described above. Since the location of the target nucleic acid within the nanochannel is recognizable by detecting the signal generated from the nanoparticle, a start point or an end point of the label bound to the target nucleic acid may be recognized and identified. Thus, location information required for determining the nucleotide sequence of the target nucleic acid may be provided.

The signal generated from the detectable label of the probe bound to the target nucleic acid may be detected by the second detector of the nucleic acid sequencing device. The signal may vary according to the types of the detectable label, and examples thereof are described above. Since the location of the target nucleic acid linked to the probe is recognizable by detecting the signal generated from the probe and it may be identified whether the probe is complementary to a specific nucleotide sequence by the signal generated by the probe. Information about location and nucleotide sequence of the target nucleic acid may be provided by the signal detected by the second detector.

According to an embodiment, the method may further include converting the signal detected from the nanoparticle into location information (e.g., positional information) of one end of the target nucleic acid and converting the signal detected from the detectable label of the probe linked to the target nucleic acid into relative location information of the end of the target nucleic acid. The location information about the probes on the target nucleic acid can be used for determining a nucleotide sequence of the target nucleic acid based on the converted information after the detecting.

That is, in the converting operation, the start point of the target nucleic acid may be determined by the signal detected from the nanoparticle and a relative location of the probe linked to the target nucleic acid may be determined by the signal detected from the probe. In addition, since the probe linked to the target nucleic acid is a nucleic acid or protein which is complementary to a specific sequence of the target nucleic acid, the nucleotide sequence of the location where the probe is detected may be detected by detecting the probe.

Meanwhile, as described with reference to the nucleic acid sequencing device, the converting is conducted by a converter, the determining of the nucleotide sequence is conducted by the calculator, and the nucleic acid sequence determined herein is output to a user by an output unit.

The present invention will be described in further detail with reference to the following examples. These examples are for illustrative purposes only and are not intended to limit the scope of the invention.

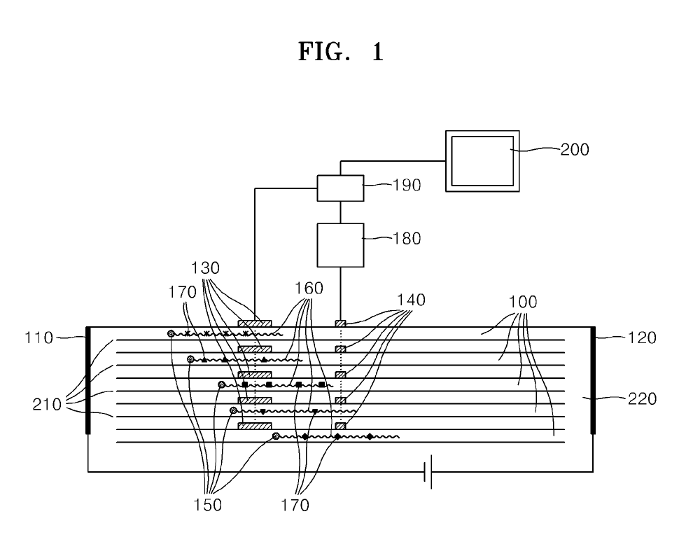

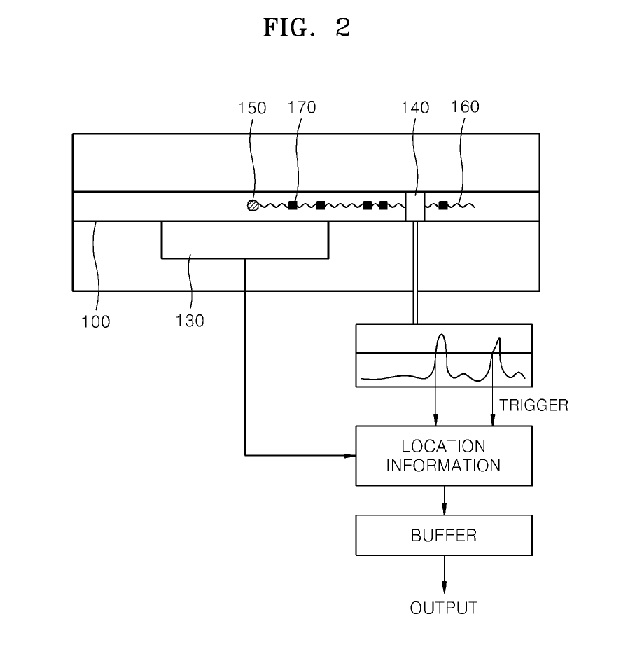

FIG. 1 is a schematic diagram of a nucleic acid sequencing device for determining a nucleotide sequence of a target nucleic acid according to an embodiment of the present invention. FIG. 2 is a schematic diagram for describing a method of determining a nucleotide sequence of a target nucleic acid using a nucleic acid sequencing device according to an embodiment of the present invention.

The method of determining the nucleotide sequence of a target nucleic acid using a nucleic acid sequencing device will be described with reference to the FIGS. 1 and 2. First, a sample including a target nucleic acid 160 is injected into a sample inlet 210 that is in fluid communication with a nanochannel 100. The target nucleic acid 160 may be linked to a nanoparticle 150 including an optically or electrically detectable label at the 5′ or 3′ end. In addition, the target nucleic acid 160 may be linked to a probe 170 that is a nucleic acid or protein complementary to the nucleotide sequence of a part of the target nucleic acid 160 and includes an optically or electrically detectable label. The width or depth of the nanochannel 100 may be in the range of about 10 to about 100 nm, and thus the target nucleic acid 160 injected into the sample inlet 210 may be restricted to being linearly stretched while flowing into the nanochannel 100.

A single strand of the target nucleic acid 160 injected into the nanochannel 100 migrates in a direction toward the second electrode 120 by negative voltage applied to a first electrode 110 and positive voltage applied to the second electrode 120. In this regard, the target nucleic acid 160 may migrate in a stretched manner within the nanochannel 100. During this locomotion of the target nucleic acid 160 in the nanochannel 100, it is linked to the nanoparticle 150. The nanoparticle 150 decelerates the migration of the target nucleic acid 160.

A first detector 130 disposed in the nanochannel 100 (or that is disposed proximate to the nanochannel 100) detects an optical or electrical signal from a detectable label on the nanoparticle 150 as the target nucleic acid 160 migrates in the nanochannel 100. A second detector 140 detects an optical or electrical signal from a detectable label of the probe 170 bound to the target nucleic acid 160. In order to obtain information of the nucleotide sequence of the target nucleic acid 160 from the detected signals, a converter 180 converts the signal (e.g., a first signal) detected by the first detector 130 into location or positional information of one end of the target nucleic acid 160. A signal (e.g., a second signal) generated from the detectable label of the probe 170 and detected by the second detector 140 is converted into relative location information of the other end of the target nucleic acid 160. Following this, a calculator 190 calculates the location information detected by the first detector 130 and converted, i.e., the start point of the target nucleic acid 160 and the relative location information of the end point of the target nucleic acid 160 that is detected by the second detector 140 and converted to determine a part of the nucleotide sequence of the target nucleic acid 160. The information of the determined nucleotide sequence may be output to a user by an output unit 200. In addition, if various types of probes 170 recognizing different nucleotide sequences are linked to each target nucleic acid 160 and the target nucleic acids 160 are passed through different nanochannels 100, the calculator 190 collects sequence information of the target nucleic acid 160 converted by the converter 180, calculates and analyzes a probe map. The probe map is an output sequence information of the entire sample of target nucleic acids 160 and is output to the user by the output unit 200.

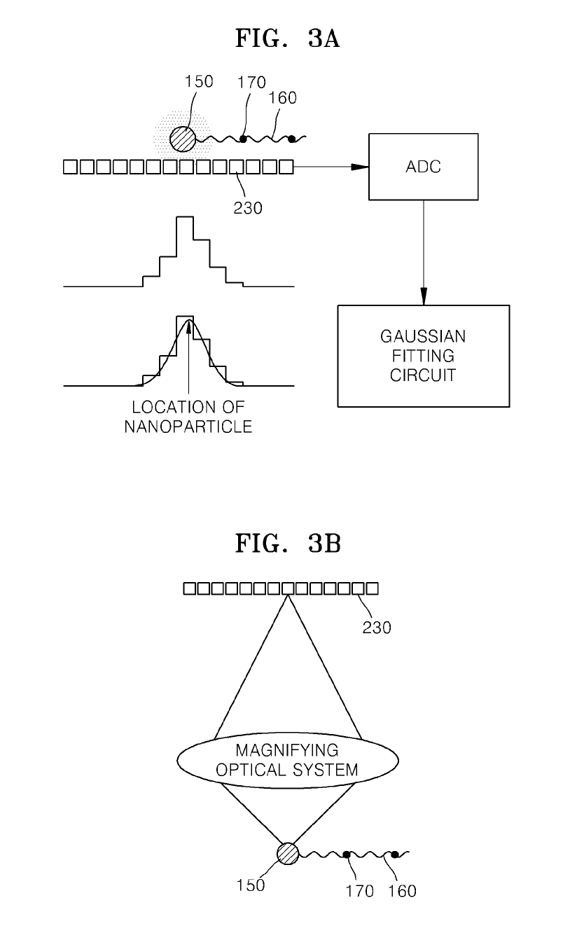

FIGS. 3A and 3B are diagrams for describing a method of measuring a location of a nanoparticle 150 linked to an end of a target nucleic acid using an image sensor as a first detector disposed in the nucleic acid sequencing device. The location of the nanoparticle 150 linked to the target nucleic acid 160 may be determined by irradiating external light on to the nanoparticle 150 and detecting a signal caused by the light (visible light, ultraviolet radiation and infrared radiation) such as fluorescence, scattering, plasmon, and absorbance that are detected by an image sensor 230. The image sensor 230 may be disposed such as to be close to the bottom of the nanochannel 100. In addition, the signal may be amplified by a magnifying optical system such as an objective lens and sensed by the image sensor 230. Light reaching a pixel of the image sensor 230 is digitized so that location (with a resolution lower than a real pixel resolution obtained by using Gaussian fitting or other methods) can be determined. Thus, the resolution of mapping may be improved.

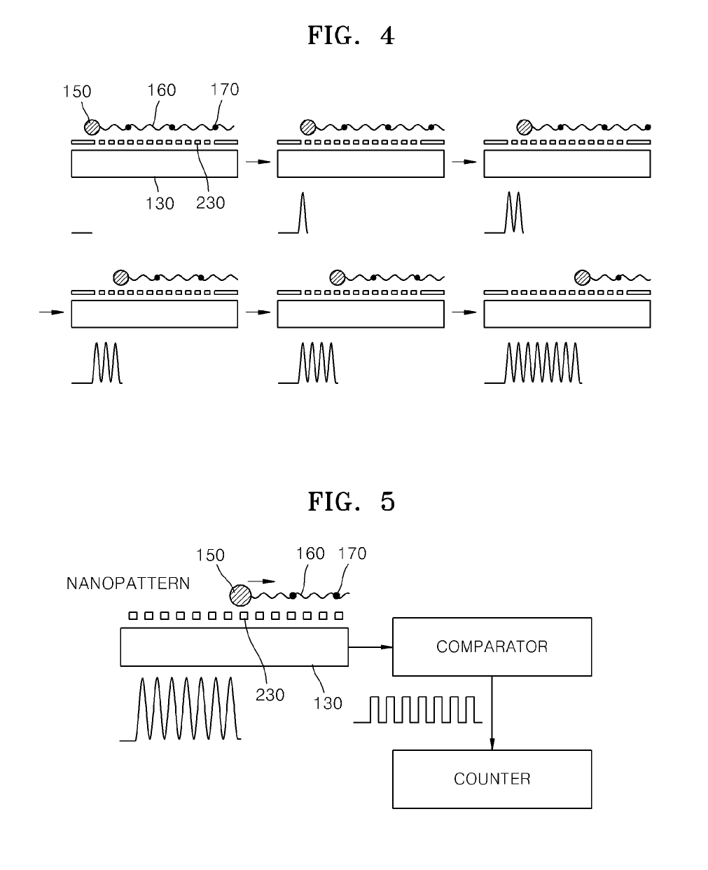

FIG. 4 is a diagram for describing a method of predicting a location of a nanoparticle using a nanopattern on a nanochannel and a first detector remotely disposed in the nucleic acid sequencing device according to an embodiment of the present invention. The nanoparticle 150 linked to one end of the target nucleic acid 160 generates a signal such as fluorescence, scattering, plasmon, and absorbance as a result of interaction with light. The amount of the signal reaching the optical detectors varies according to locations of the nanoparticle 150. In addition, a nanopattern in the nanochannel 100 is formed at a distance in the range of about 1 to about 100 nm from openings and has at intervals in the range of about 10 to about 500 nm. As the nanoparticle 150 migrates, signal events occur, and thus the number of signal events is related to the migrating distance of the nanoparticle 150. Thus, the migrating distance of the nanoparticle 150 may be obtained by measuring a series of sequential signal events.

FIG. 5 is a diagram for describing a method of converting a signal in a converter to determine a location of a nanoparticle linked to a target nucleic acid in the nucleic acid sequencing device according to an embodiment of the present invention. The signal detected by the first detector 130 is converted into a pulse signal by a circuit such as a comparator and counted by a counter. The counted value is transmitted to the calculator 190 with information obtained by converting the signal from the probe detected by the second detector 140. The second detector 140 may include a nano sensor.

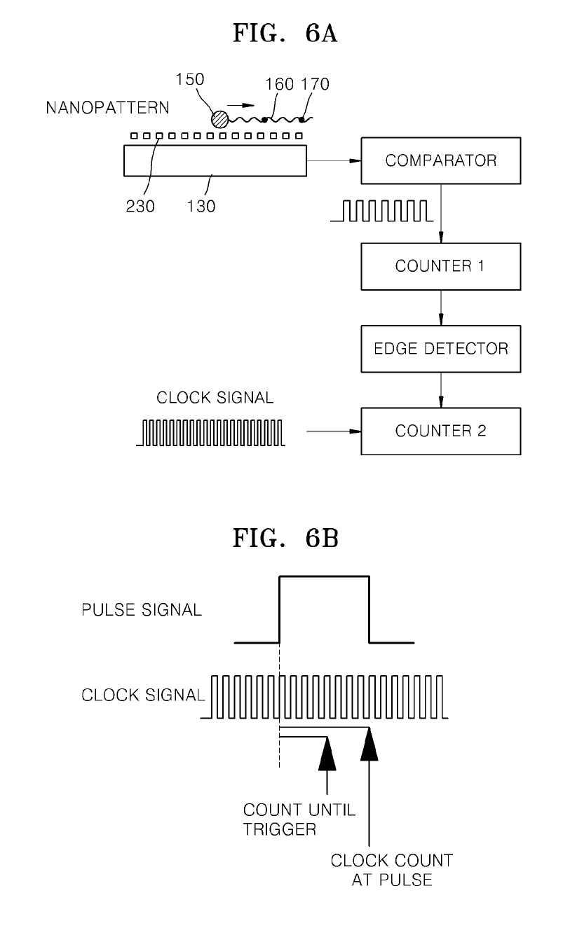

FIGS. 6A and 6B are diagrams for describing a method of improving precision of the measurement of a location of a nanoparticle linked to a target nucleic acid in the nucleic acid sequencing device according to an embodiment of the present invention. The location of the nanoparticle 150 may be precisely detected by dividing the interval of the nanopattern. For example, a pulse is divided into a number of segments using separate clock signals, e.g., a clock count of the time to a subsequent pulse edge when the probe 170 is detected and a clock count of the time when the probe 170 is detected to precisely measure the location of the nanoparticle 150. Furthermore, accuracy of the location measurement may be improved by dividing the pulse signal.

As described above, according to the nucleic acid sequencing device and the method of determining a nucleotide sequence of a target nucleic acid using the same according to the one or more of the above embodiments of the present invention, the nucleotide sequence of the target nucleic acid may be efficiently determined.

It should be understood that the exemplary embodiments described therein should be considered in a descriptive sense only and not for purposes of limitation. Descriptions of features or aspects within each embodiment should typically be considered as available for other similar features or aspects in other embodiments.

What is claimed is:

1. A nucleic acid sequencing device comprises:
at least one nanochannel,
a first electrode and a second electrode disposed at opposite ends of the nanochannel for applying a voltage in the lengthwise direction of the nanochannel, and
a first detector that detects a location signal of a target nucleic acid passing through the nanochannel and a second detector that detects a signal from a detectable label bound to the target nucleic acid.

2. The nucleic acid sequencing device of claim 1, wherein the nanochannel has a width or depth in the range of about 10 to about 1,000 nanometers.

3. The nucleic acid sequencing device of claim 1, wherein the first detector is an optical detector or an electrical detector.

4. The nucleic acid sequencing device of claim 1, wherein the second detector is an optical detector or an electrical detector.

5. The nucleic acid sequencing device of claim 3, wherein the electrical detector detects at least one selected from the group consisting of a current, voltage, resistance, and impedance.

6. The nucleic acid sequencing device of claim 4, wherein the electrical detector detects at least one selected from the group consisting of a current, voltage, resistance, and impedance.

7. The nucleic acid sequencing device of claim 3, wherein the optical detector detects at least one selected from the group consisting of an absorbance, transmission, scattering, fluorescence, fluorescence, resonance energy transfer, surface plasmon resonance, surface enhanced Raman scattering, and diffraction.

8. The nucleic acid sequencing device of claim 1, further comprising a converter that converts the signal detected from of one end of the target nucleic acid by the first detector into location information of the one end of the target nucleic acid and the signal detected by the second detector into relative location information of the probes on the target nucleic acid; and a calculator that determines a nucleotide sequence of the target nucleic acid based on the information obtained from the converter.

9. The nucleic acid sequencing device of claim 8, further comprising an output unit that outputs the nucleotide sequence of the target nucleic acid determined by the calculator to a user.

10. The nucleic acid sequencing device of claim 1, further comprising a sample inlet and a sample outlet which are connected to openings of the both ends of the nanochannel in a fluid communicable manner.

11. The nucleic acid sequencing device of claim 10, wherein the sample inlet comprises a microfluidic region comprising at least one microfluidic channel and a nanofluidic region comprising at least one nanofluidic channel so as to allow fluid flow therebetween.

12. A method of determining a nucleotide sequence of a target nucleic acid, the method comprising:
linking a nanoparticle comprising a detectable label to a 5′ or 3′ end of a target nucleic acid having a nucleotide sequence to be detected;
making the target nucleic acid contact a probe comprising a detectable label;
injecting the target nucleic acid into a nanochannel; applying a voltage between the first electrode and the second electrode of the nucleic acid sequencing device; and
detecting a signal generated from the nanoparticle linked to the target nucleic acid and a signal generated from the detectable label of the probe bound to the target nucleic acid.

13. The method of claim 12, wherein the diameter of the nanoparticle is about 1 to about 200 nm.

14. The method of claim 12, wherein the target nucleic acid is single-stranded or double stranded.

15. The method of claim 12, wherein the probe is a nucleic acid or protein that is complementary to a part of the nucleotide sequence of the target nucleic acid.

16. The method of claim 12, wherein the detectable label is selected from the group consisting of a colored bead, antigen determinant, enzyme, hybridizable nucleic acid, chromophore, fluorescent material, electrically detectable material, material providing modified fluorescence-polarization or modified light-diffusion, and quantum dot.

17. The method of claim 12, further comprising
converting the signal detected from the nanoparticle into location information of one end of the target nucleic acid and converting the signal detected from the detectable label of the probe bound to the target nucleic acid into relative location information of the other end of the target nucleic acid; and
determining the nucleotide sequence of the target nucleic acid based on the converted information after the detecting.
